(19) United States
(12) Patent Application Publication
(10) Pub. No.: US 2024/0382450 A1
(43) Pub. Date: Nov. 21, 2024

(54) COMPOSITION OF B-CARYOPHYLLENE AND A STATIN, AND METHODS OF USING SAME

(71) Applicant: OrthoTreat Ltd., Tel Aviv Jaffa (IL)

(72) Inventors: Hilik MAROM, Har Adar (IL); Tami EHRMANN BARR, Meitar (IL)

(73) Assignee: OrthoTreat Ltd., Tel Aviv Jaffa (IL)

(21) Appl. No.: 18/683,767

(22) PCT Filed: Aug. 17, 2022
(86) PCT No.: PCT/IL2022/050898

§ 371 Date: Feb. 14, 2024

Related U.S. Application Data
(60) Provisional application No. 63/234,423, filed on Aug. 18, 2021.

(51) Int. Cl. A61K 31/366; A61K 31/015; A61P 19/08

(52) U.S. Cl. CPC A61K 31/366; A61K 31/015; A61P 19/08

(57) ABSTRACT

The present invention, in some embodiments, is directed to a pharmaceutical composition including β-caryophyllene and a statin, and use of same, such as in a method for treating a subject afflicted with a bone-related disease or a disorder associated therewith.

### CROSS-REFERENCE TO RELATED APPLICATIONS

This application claims the benefit of priority of U.S. Provisional Patent Application No. 63/234,423, titled “COMPOSITION OF B-CARYOPHYLLENE AND A STATIN, AND METHODS OF USING SAME”, filed 18 Aug. 2021, the contents of which are incorporated herein by reference in their entirety.

### FIELD OF THE INVENTION

The present invention is in the field of orthopedics, and particularly relates to pharmaceutical compositions comprising β-caryophyllene and one or more statins, for use in improving bone healing process in a subject in need thereof.

### BACKGROUND

Bone fractures of all types are among the most common injuries affecting millions of individuals of all ages and both genders worldwide. Healing of bone fractures is affected by multiple environmental, systemic, local, and pharmacological factors. Fracture healing involves a complex and sequential set of events to restore injured bone to pre-fracture condition, including three main stages of fracture healing, inflammation, repair and remodeling.

Statins, also known as HMG-CoA (3-hydroxy-3-methylglutaryl-CoA) reductase inhibitors, have been widely prescribed for cardiovascular disease (CVD) for decades. Nonetheless, several studies have demonstrated that statins exert bone anabolic effect and may be helpful for the treatment of osteoporosis. Several experiments have analyzed the mechanisms of bone anabolism regulated by statins. Substantial clinical trials data demonstrate statin safety and efficacy in both men and women.

(−)-β-caryophyllene (β-caryophyllene) is a natural bicyclic sesquiterpene that is a constituent of many essential oils, especially oil from the stems and flowers of Syzygium aromaticum (cloves), the essential oil of Cannabis sativa and hops. β-caryophyllene is notable for having a cyclobutane ring, as well as a trans-double bond in a 9-membered ring, both rarities in nature. Caryophyllene is one of the chemical compounds that contributes to the aroma of black pepper.

Following bone fracture, the haematoma acts as a scaffold for recruited polymorphonuclear neutrophils (PMNs) to clear the area of dead cells and debris and secrete chemokines, notably CCL2 (chemokine ligand 2) and IL-6, to attract macrophages. These PMNs exert their effects and die quickly. Osteomacs and recruited macrophages are pivotal for intramembranous and endochondral ossification, respectively. Following macrophages polarization (M1/M2 phenotype) lymphocytes migrate to the fracture site, initiate the adaptive immune response, and secrete proinflammatory cytokines, such as IL-1, IL-6, tumor necrosis factor-α (TNF-α) and receptor activator of nuclear factor kappa-B ligand (RANKL). Inhibition of this inflammatory phase disrupts bone formation and increases the risk of non-union. On the other hand, it is well known that systemic inflammation, as observed in patients with rheumatoid arthritis, diabetes mellitus, multiple trauma or sepsis, increase fracture healing time and the rate of complications, including non-unions, also due to the lack of anti-inflammatory cytokines such as IL-4, IL-10, and IL-13 that facilitate bone formation. Moreover, during inflammation-period, blood flow to the fracture area is disturbed.

β-Caryophyllene modulates expression and activity of tumor necrosis factor alpha (TNF-α), where TNF-α exert opposite effects depending on the context in which it is released. Stimulation of the molecular signaling pathway responsible for TNF-α production induces osteogenic differentiation of mesenchymal stem cells in vitro, while signals that suppress TNF-α decrease osteogenic differentiation. TNF-α regulates the differentiation and function of both osteoblasts and osteoclasts via the two cell-surface receptors for TNF-α Tumor Necrosis Factor Receptors TNFR1 (always present in bone tissue) and TNFR2 (only expressed following bone injury). Indeed, TNF-α signaling promoted bone formation in cells from both normal and TNFR1-deficient mice while the opposite effect was observed with TNFR2-deficient cells, where TNF-α signaling stimulated osteoclast differentiation and bone resorption.

β-caryophyllene possesses protective effects on brain damage and chemical induced seizure. β-caryophyllene restores the activity of catalase (CAT), superoxide dismutase (SOD), and glutathione peroxidase (GPx), activates cannabinoid receptor 2(CB2) and the peroxisome proliferator-activated receptor-γ (PPARγ) pathway, preventing the Alzheimer-like phenotype in APP/PS1 mice.

There is still a great need for pharmaceutical compositions having superior or improved bone healing characteristics, and methods of using same.

### SUMMARY

According to a first aspect, there is provide a pharmaceutical composition comprising β-caryophyllene and a statin, for use in the treatment or amelioration of a bone-related disease or a disorder associated therewith, in a subject in need thereof.

According to another aspect, there is provided a method of treating a subject afflicted with a bone-related disease or a disorder associated therewith, comprising administering to the subject a therapeutically effective amount of a pharmaceutical composition comprising β-caryophyllene and a statin.

In some embodiments, the pharmaceutical composition comprises β-caryophyllene and a statin in a weight per weight (w/w) ratio ranging from 10:1 (w/w) to 1:10 (w/w).

In some embodiments, the statin is a hydrophilic statin or a lipophilic statin.

In some embodiments, the statin is selected from the group consisting of: lovastatin, simvastatin, fluvastatin, rosuvastatin, pravastatin, pitavastatin, and any combination thereof.

In some embodiments, the statin is lovastatin or simvastatin.

In some embodiments, β-caryophyllene is present in the pharmaceutical composition as a highly purified extract of Cannabis.

In some embodiments, β-caryophyllene is synthetically- or semi-synthetically produced.

In some embodiments, the pharmaceutical composition further comprises cannabidiol (CBD).

In some embodiments, the bone-related disease or a disorder associated therewith comprises a bone fracture.

In some embodiments, the subject is in need of any one of: dental sinus lift, dental graft, and both.

In some embodiments, treating comprises: reducing fracture length per bone width, increasing maximal load, increasing stiffness, or any combination thereof, of a bone of the subject.

In some embodiments, treating comprises increasing number of bone proliferating cells, rate of bone cell proliferation, or both, in the subject.

In some embodiments, treating comprises increasing: endosteal and periosteal proliferation, defect fill, bone quality, or any combination thereof, in a bone of the subject.

In some embodiments, the administering comprises locally administering.

In some embodiments, the administering comprises intraoperative administration.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIG. 1 includes a vertical bar graph showing reduced fracture length following 35 treatment days of femoral bone fracture with Lovastatin and β-caryophyllene (1:10; weight per weight (w/w)), hereinafter Group 1 (G1).

FIG. 2 includes a vertical bar graph showing reduced fracture length following 35 treatment days of femoral bone fracture with Simvastatin(S) and β-caryophyllene (B; 1:10 w/w), hereinafter Group 2 (G2).

FIG. 3 includes a vertical bar graph showing reduced fracture length following 35 treatment days of femoral bone fracture with Simvastatin and cannabidiol (CBD; 1:10 w/w), hereinafter Group 3 (G3).

FIG. 4 includes a vertical bar graph showing reduced fracture length following 35 treatment days of femoral bone fracture with Lovastatin and CBD (1:10 w/w), hereinafter Group 4 (G4).

FIG. 5 includes a vertical bar graph showing the greatest increase in bone maximal load following 35 treatment days of femoral bone fracture was achieved with Lovastatin (L) and β-caryophyllene (B), i.e., G1. CBD—C; Simvastatin—S.

FIG. 6 includes a vertical bar graph showing that the greatest increase in bone stiffness following 35 treatment days of femoral bone fracture was achieved with Lovastatin (L) and β-caryophyllene (B), i.e., G1.

FIG. 7 includes a vertical bar graph showing bone proliferation following 35 treatment days of femoral bone fracture with β-caryophyllene following decalcification. R(T)—right leg being treated; L—untreated left leg.

FIG. 8 includes a vertical bar graph showing bone endosteal and periosteal proliferation, defect fill, and bone quality, which were evaluated following 35 treatment days of femoral bone fracture with β-caryophyllene (none-decalcification).

FIG. 9 includes a vertical bar graph showing that increase in energy to maximal load (mj) following 35 treatment days of femoral bone fracture was achieved with Lovastatin (L) and β-caryophyllene (B), i.e., G1, as well with Lovastatin (L) and CBD (C), i.e., G3.

FIGS. 10A-10B include plates layout for cytotoxicity test in osteoblasts/osteoclasts and MUTZ-3 cells. Toxicity effect was tested in the presence of PBS, β-caryophyllene, or Lovastatin (10A), or in the presence of PBS or lovastatin and β-caryophyllene at the indicated concentrations (10B).

FIGS. 11A-11D include graphs showing light intensity (fluorescence intensity at 485-500 nm_{Ex}/520-530 nm_{Em}) measured on osteoblasts culture with β-caryophyllene concentrations ranging from 0 to 6,561 μM with lovastatin at concentrations of 0, 1, 10 and 100 μM after 24 hours (11A) and 96 hours (11B) and on MUTZ-3 culture after 24 hours (11C) and 96 hours (11D).

FIG. 12 includes an illustration of a non-limiting plate layout for determining the differentiation of human mesenchymal stem cells (hMSC) into osteoblasts.

FIG. 13 includes a non-limiting scheme depicting a process for obtaining of bone explant from the mice tibia, following Maeda et al., 2018, Journal of Biomechanics.

FIG. 14 includes an illustration of a non-limiting plate layout for evaluating the effects of β-caryophyllene and lovastatin on calcification of bone specimens in culture.

### DETAILED DESCRIPTION

### Pharmaceutical Composition

According to some embodiments, there is provided a pharmaceutical composition comprising β-caryophyllene and a statin, for use in the treatment or amelioration of a bone-related disease or a disorder associated therewith, in a subject in need thereof.

In some embodiments, the pharmaceutical composition comprises β-caryophyllene and a statin in a weight per weight (w/w) ratio ranging from: 1,000:1 (w/w) to 1:1,000 (w/w), 800:1 (w/w) to 1:800 (w/w), 700:1 (w/w) to 1:700 (w/w), 600:1 (w/w) to 1:600 (w/w), 500:1 (w/w) to 1:500 (w/w), 350:1 (w/w) to 1:350 (w/w), 200:1 (w/w) to 1:200 (w/w), 150:1 (w/w) to 1:150 (w/w), 100:1 (w/w) to 1:100 (w/w), 80:1 (w/w) to 1:80 (w/w), 70:1 (w/w) to 1:70 (w/w), 60:1 (w/w) to 1:60 (w/w), 50:1 (w/w) to 1:50 (w/w), 35:1 (w/w) to 1:35 (w/w), 20:1 (w/w) to 1:20 (w/w), 15:1 (w/w) to 1:15 (w/w), 10:1 (w/w) to 1:10 (w/w), 9:1 (w/w) to 1:9 (w/w), 8:1 (w/w) to 1:8 (w/w), 7:1 (w/w) to 1:7 (w/w), 6:1 (w/w) to 1:6 (w/w), 5:1 (w/w) to 1:5 (w/w), 4:1 (w/w) to 1:4 (w/w), 3:1 (w/w) to 1:3 (w/w), or 2:1 (w/w) to 1:2 (w/w). Each possibility represents a separate embodiment of the invention.

In some embodiments, the pharmaceutical composition comprises β-caryophyllene and a statin in a mole per mole (m/m) ratioranging from: 1,000:1 (m/m) to 1:1,000 (m/m), 800:1 (m/m) to 1:800 (m/m), 700:1 (m/m) to 1:700 (m/m), 600:1 (m/m) to 1:600 (m/m), 500:1 (m/m) to 1:500 (m/m), 350:1 (m/m) to 1:350 (m/m), 200:1 (m/m) to 1:200 (m/m), 150:1 (m/m) to 1:150 (m/m), 100:1 (m/m) to 1:100 (m/m), 80:1 (m/m) to 1:80 (m/m), 70:1 (m/m) to 1:70 (m/m), 60:1 (m/m) to 1:60 (m/m), 50:1 (m/m) to 1:50 (m/m), 35:1 (m/m) to 1:35 (m/m), 20:1 (m/m) to 1:20 (m/m), 15:1 (m/m) to 1:15 (m/m), 10:1 (m/m) to 1:10 (m/m), 9:1 (m/m) to 1:9 (m/m), 8:1 (m/m) to 1:8 (m/m), 7:1 (m/m) to 1:7 (m/m), 6:1 (m/m) to 1:6 (m/m), 5:1 (m/m) to 1:5 (m/m), 4:1 (m/m) to 1:4 (m/m), 3:1 (m/m) to 1:3 (m/m), or 2:1 (m/m) to 1:2 (m/m). Each possibility represents a separate embodiment of the invention.

In some embodiments, a statin is a hydrophilic statin or a lipophilic statin.

In some embodiments, a statin is selected from: lovastatin, simvastatin, fluvastatin, rosuvastatin, pravastatin, pitavastatin, or any combination thereof.

In some embodiments, a statin is lovastatin. In some embodiments a statin is simvastatin.

In some embodiments, the pharmaceutical composition comprises β-caryophyllene and lovastatin. In some embodiments, the pharmaceutical composition comprises β-caryophyllene and simvastatin.

In some embodiments, the pharmaceutical composition comprises a plurality of statins.

As used herein, the term “plurality” refers to any integer equal to or greater than 2.

In some embodiments, a plurality of statins comprises at least one hydrophilic stain and at least one lipophilic statin.

In some embodiments, a plurality of statins comprises a plurality of different types of hydrophilic statins.

In some embodiments, a plurality of statins comprises a plurality of different types of lipophilic statins.

In some embodiments, β-caryophyllene is present in the pharmaceutical composition as a highly purified extract of Cannabis.

In some embodiments, β-caryophyllene is synthetically- or semi-synthetically produced.

In one embodiment, β-caryophyllene is extracted from a plant or plant material. In one embodiment, β-caryophyllene is extracted from a Cannabis plant or a Cannabis material. In one embodiment, β-caryophyllene is chemically synthesized. In one embodiment, β-caryophyllene is biologically synthesized and/or is biosynthetic β-caryophyllene.

As used herein, the term “biosynthetic β-caryophyllene” refers to β-caryophyllene being produced by microbes, such as, but not limited to, by means of fermentation.

As used herein, the terms “caryophyllene” and “β-caryophyllene” refer to natural bicyclic sesquiterpenes. In some embodiments, β-caryophyllene includes or comprises α-caryophyllene (humulene). In some embodiments, β-caryophyllene includes or comprises isocaryophyllene.

In some embodiments, β-caryophyllene comprises or is characterized by combined modulation of secretion of proinflammatory cytokines, tumor necrosis factor alpha (TNF-α), and specific agonist activity of cannabinoid receptor 2 (CB2).

In some embodiments, β-caryophyllene comprises or is characterized by modulation of secretion of proinflammatory cytokines.

In some embodiments, β-caryophyllene comprises or is characterized by modulation of TNF-α.

In some embodiments, β-caryophyllene comprises or is characterized by specific agonist activity of CB2.

As used herein, the terms “modulate” or “modulation” refer to the ability to increase or reduce the expression and/or effectivity and/or efficacy of a substance, a group of substances, a cascade of progresses and events (e.g., signaling), or any combination thereof.

In some embodiments, β-caryophyllene is water soluble β-caryophyllene. In some embodiments, β-caryophyllene is in the form of an oil. In some embodiments, β-caryophyllene in in the form of oil-in-water. In some embodiments, β-caryophyllene is in the form of crystals.

In some embodiments, β-caryophyllene comprises any derivative of β-caryophyllene, or any plurality thereof, as long as the derivative comprises or is characterized by the activity of β-caryophyllene as disclosed herein.

The term “derivative” is intended to mean any one of natural caryophilane, synthetic caryophilane, natural humulane, synthetic humulane and any functional analogs thereof having activity of β-caryophyllene as disclosed herein.

As used herein, the term “statin” encompasses any 3-hydroxy-3-methyl-glutaryl-coenzyme A (HMG-COA) reductase inhibitor.

In some embodiments, the pharmaceutical composition of the invention further comprises a cannabinoid. In some embodiments, the cannabinoid is or comprises cannabidiol (CBD).

In one embodiment, CBD is extracted from a plant or plant material. In one embodiment, CBD is extracted from a Cannabis plant or a Cannabis material. In one embodiment, CBD is chemically synthesized. In one embodiment, CBD is biologically synthesized and/or is biosynthetic CBD.

As used herein, the term “biosynthetic CBD” refers to CBD being produced by microbes, such as, but not limited to, by means of fermentation.

As used herein, the term “carrier,” “excipient,” or “adjuvant” refers to any component of a pharmaceutical composition that is not the active agent. As used herein, the term “pharmaceutically acceptable carrier” refers to non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations. Some non-limiting examples of substances which can serve as a carrier herein include sugar, starch, cellulose and its derivatives, powered tragacanth, malt, gelatin, talc, stearic acid, magnesium stearate, calcium sulfate, vegetable oils, polyols, alginic acid, pyrogen-free water, isotonic saline, phosphate buffer solutions, cocoa butter (suppository base), emulsifier (e.g. carbomer, hydroxypropyl cellulose, sodium lauryl sulfate) as well as other non-toxic pharmaceutically compatible substances used in other pharmaceutical formulations. Wetting agents and lubricants such as sodium lauryl sulfate, as well as coloring agents, flavoring agents, excipients, stabilizers, antioxidants, and preservatives may also be present. Any non-toxic, inert, and effective carrier may be used to formulate the compositions contemplated herein. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide,” U.S. Food and Drug Administration (FDA) Center for Drug Evaluation and Research (CDER) Office of Management, the contents of all of which are hereby incorporated by reference in their entirety. Examples of pharmaceutically acceptable excipients, carriers and diluents useful in the present compositions include distilled water, physiological saline, Ringer's solution, dextrose solution, Hank's solution, and DMSO. These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990); Remington's Pharmaceutical Sciences, 18th Ed., Mack Publishing Co., Easton, Pa. (1990); and Remington: The Science and Practice of Pharmacy, 21st Ed., Lippincott Williams & Wilkins, Philadelphia, Pa., (2005), each of which is incorporated by reference herein in its entirety. The presently described composition may also be contained in artificially created structures such as liposomes, ISCOMS, slow-releasing particles, and other vehicles which increase the half-life of the peptides or polypeptides in serum. Liposomes include emulsions, foams, micelles, insoluble monolayers, liquid crystals, phospholipid dispersions, lamellar layers and the like. Liposomes for use with the presently described peptides are formed from standard vesicle-forming lipids which generally include neutral and negatively charged phospholipids and a sterol, such as cholesterol. The selection of lipids is generally determined by considerations such as liposome size and stability in the blood. A variety of methods are available for preparing liposomes as reviewed, for example, by Coligan, J. E. et al, Current Protocols in Protein Science, 1999, John Wiley & Sons, Inc., New York, and see also U.S. Pat. Nos. 4,235,871, 4,501,728, 4,837,028, and 5,019,369.

The carrier may comprise, in total, from about 0.1% to about 99.99999% by weight of the pharmaceutical compositions presented herein.

A pharmaceutically-acceptable carrier suitable for the preparation of unit dosage form of a composition as described herein for peroral administration is well-known in the art.

In some embodiments, the pharmaceutical composition further comprises binders (e.g. acacia, cornstarch, gelatin, carbomer, ethyl cellulose, guar gum, hydroxypropyl cellulose, hydroxypropyl methyl cellulose, povidone), disintegrating agents (e.g. cornstarch, potato starch, alginic acid, silicon dioxide, croscarmellose sodium, crospovidone, guar gum, sodium starch glycolate), additives such as albumin or gelatin to prevent absorption to surfaces, detergents (e.g., Tween 20, Tween 80, Pluronic F68, bile acid salts), protease inhibitors, surfactants (e.g. sodium lauryl sulfate), permeation enhancers, solubilizing agents (e.g., glycerol, polyethylene glycerol), stabilizers (e.g. hydroxypropyl cellulose, hydroxypropylmethyl cellulose), viscosity increasing agents (e.g. carbomer, colloidal silicon dioxide, ethyl cellulose, guar gum lubricants (e.g. stearic acid, magnesium stearate, polyethylene glycol, sodium lauryl sulfate), flow-aids (e.g. colloidal silicon dioxide), plasticizers (e.g. diethyl phthalate, triethyl citrate), polymer coatings (e.g., poloxamers or poloxamines), and/or coating and film forming agents (e.g. ethyl cellulose, acrylates, polymethacrylates).

In some embodiments, preparation of effective amount or dose can be estimated initially from in vitro assays. In one embodiment, a dose can be formulated in animal models, and such information can be used to more accurately determine useful doses in humans.

In one embodiment, toxicity and therapeutic efficacy of the active ingredients described herein can be determined by standard pharmaceutical procedures in vitro, in cell cultures or experimental animals. In one embodiment, the data obtained from these in vitro and cell culture assays and animal studies can be used in formulating a range of dosage for use in human. In one embodiment, the dosages vary depending upon the dosage form employed and the route of administration utilized. In one embodiment, the exact formulation, route of administration and dosage can be chosen by the individual physician in view of the patient's condition. [See e.g., Fingl, et al., (1975) “The Pharmacological Basis of Therapeutics”, Ch. 1 p.1].

In some embodiments, the disclosed composition is formulated for oral administration. For oral applications, the composition may be in the form of tablets, caplets or capsules, which can contain any of the ingredients, or compounds mentioned hereinabove. When the dosage unit form is a capsule, it can contain, in addition to materials of the above type, a liquid carrier, such as fatty oil. In addition, dosage unit forms can contain various other materials which modify the physical form of the dosage unit. A tablet comprising the disclosed composition can further be film coated. In some embodiments, oral application of the composition may be in the form of an edible product, such as a chewable tablet.

In some embodiments, the composition is formulated as a nutraceutical composition, a pharmaceutical composition, a cosmeceutical composition, a dietary supplement, or any combination thereof.

For a non-limiting example, the composition may be incorporated in dry formulations of nutritional supplements and packaged in gel capsules, tablets, sachets and the like. In yet another example, the product may be useful in a liquid form or packaging in soft capsules.

### Method of Treatment

According to some embodiments, there is provided a method of treating a subject afflicted with a bone-related disease or a disorder associated therewith, comprising administering to the subject a therapeutically effective amount of a pharmaceutical composition comprising β-caryophyllene and a statin.

As used herein, the term “bone-related disease or a disorder associated therewith” encompasses any disease or disorder wherein bone formation, deposition, or resorption is abnormal. In some embodiments, a bone-related disease, as disclosed herein is characterized by or comprises excessive angiogenesis.

In some embodiments, a bone-related disease, as used herein, comprises a joint-related disease or disorder.

In some embodiments, a bone-related disease, as used herein, comprises a dental-related disease or disorder.

In some embodiments, a bone-related disease or a disorder associated therewith comprises or is characterized by inflammation of a bone tissue, of a joint tissue, of a dental tissue, or any combination thereof.

In some embodiments, the bone-related disease or a disorder associated therewith comprises a bone fracture.

In some embodiments, treating comprises: reducing fracture length per bone width, increasing maximal load, increasing stiffness, or any combination thereof, of a bone of the subject.

Methods for determining fracture length per bone width, bone maximal load, bone stiffness, are common and would be apparent to one of ordinary skill in the art. Non-limiting examples for such methods include, but are not limited to, quantitative CT (computed tomography), high-resolution peripheral quantitative CT, micro-CT, HRMRI (high-resolution magnetic resonance imaging), HRpQCT (high-resolution peripheral quantitative computed tomography), NMR (nuclear magnetic resonance imaging), qBEI (quantitative backscattered electron imaging), QCT (quantitative computed tomography), RPI (reference point indentation), SAXS (small-angle x-ray scattering), SEM (scanning electron microscopy), TGA (thermogravimetric analysis), XRD (x-ray diffraction), microbeam testing, microindentation and nanoindentation, some of which, are exemplified hereinbelow.

In some embodiments, treating comprises increasing the number of bone proliferating cells, rate of bone cell proliferation, or both, in a bone of subject.

Methods for determining cells proliferation, are common and would be apparent to one of ordinary skill in the art. Non-limiting examples for such methods include, but are not limited to, immunoassays using biomarkers of cell proliferation, e.g., Ki67, utilized in flow cytometry, immunocytochemistry, or others.

In some embodiments, treating comprises increasing: endosteal and periosteal proliferation, defect fill, bone quality, or any combination thereof, in a bone of the subject.

Methods for determining endosteal and periosteal proliferation, defect fill, bone quality, are common and would be apparent to one of ordinary skill in the art. Non-limiting examples for such methods include, but are not limited to, staining; decalcification, labelling and microscopic observations and gene and protein expression, some of which are exemplified hereinbelow.

In some embodiments, treating comprises reducing bone inflammation, joint inflammation, or both. In some embodiments, treating comprises increasing the number, proliferation rate, differentiation rate, activation rate, activity, or any combination thereof, of osteoblasts in the subject.

As used herein, the term “osteoblast activity” refers to the cellular activity of osteoblasts to synthesize the collagenous precursors of bone extracellular matrix, regulate mineralization of the matrix to form bone (e.g., bone formation and mineralization), as well as their function in bone remodeling and reformation. The mineralization of bone occurs by deposition of carbonated hydroxyapatite crystals in an extracellular matrix consisting of type I collagen and a variety of non-collagenous proteins.

As used herein, an “osteoblast” is a bone-forming cell that is derived from mesenchymal osteoprogenitor cells and forms an osseous matrix in which it becomes enclosed as an osteocyte. A mature osteoblast is one capable of forming bone extracellular matrix in vivo and can be identified in vitro by its capacity to form mineralized nodules which reflects the generation of extracellular matrix. An immature osteoblast is not capable of forming mineralized nodules in vitro.

In some embodiments, treating comprises reducing the number, proliferation rate, differentiation rate, activation rate, activity, or any combination thereof, of osteoclasts in the subject.

As used herein, an “osteoclast” is a large multinucleated cell with abundant acidophilic cytoplasm derived from hematopoietic stem cells, functioning in the absorption and removal of osseous tissue. Osteoclasts become highly active in the presence of parathyroid hormone, causing increased bone resorption and release of bone salts (phosphorus and, especially, calcium) into the extracellular fluid. Osteoclasts are also identified based on the formation of actin ring structure and a polar cell body during resorption, and contraction in response to calcitonin. A mature osteoclast, but not its precursor cell, can be identified based on the secretion of the enzyme, Tartrate-resistant Acidic Phosphatase (TRAP).

In some embodiments, treating comprises increasing the ratio of osteoblasts to osteoclasts in the subject. In some embodiments, treating comprises increasing the ratio of active osteoblasts to active osteoclasts in the subject. In some embodiments, treating comprises increasing the ratio of differentiated and/or activated osteoblasts to differentiated and/or activated osteoclasts in the subject.

In some embodiments, treating comprises bone healing.

In some embodiments, any one of: the number, proliferation rate, differentiation rate, activation rate, activity, or any combination thereof, of osteoblasts, osteoclasts, or both, is determined at a site in need of treatment as disclosed herein, in the subject, e.g., a damaged: bone tissue, joint tissue, or dental tissue.

In some embodiments, the determining is in a sample obtained or derived from the subject. In some embodiments, the determining is performed in vitro or ex vivo.

In some embodiments, a bone is a femur of a subject.

In some embodiments, administering comprises locally administering. In some embodiments, the pharmaceutical composition disclosed herein is formulated for a local administration. In some embodiments, the pharmaceutical composition disclosed herein is formulated for a controlled-or a slow-release. In some embodiments, the administering comprises intraoperative administration. In some embodiments, the administering comprises locally administering the pharmaceutical composition disclosed herein during an operation or surgery (i.e., intraoperative) of the subject.

In some embodiments, the subject is mammal subject. In some embodiments, the subject is a human subject. In some embodiments, the subject is afflicted with or at increased risk of developing a bone-related disease or a disorder associated therewith. In some embodiments, the subject is in need of: dental sinus lift, dental graft, or both. In some embodiments, the subject is afflicted with a bone fracture.

In some embodiments, the reduce, reducing, inhibit, or inhibiting is at least 5%, 10%, 25%, 30%, 40%, 50%, 60%, 70%, 80%, 90%, 95%, or 100% reduction or inhibition compared to a control, or any value and range therebetween. Each possibility represents a separate embodiment of the invention.

In some embodiments, increase, increasing, enhance, or enhancing is at least 5%, 10%, 35%, 50%, 80%, 100%, 150%, 270%, 400%, 650%, 800%, or 1,000% increase compared to a control, or any value and range therebetween. Each possibility represents a separate embodiment of the invention.

As used herein, the terms “treatment” or “treating” of a disease, disorder or condition encompasses alleviation of at least one symptom thereof, a reduction in the severity thereof, or inhibition of the progression thereof. Treatment need not mean that the disease, disorder, or condition is totally cured. To be an effective treatment, a useful composition herein needs only to reduce the severity of a disease, disorder, or condition, reduce the severity of symptoms associated therewith, or provide improvement to a patient or subject's quality of life.

As used herein, “treating” comprises ameliorating and/or preventing.

In some embodiments, the combination of β-caryophyllene and one or more statins may be referred to in the application as “the combination” and/or “the formulation” and/or the “active pharmaceutical ingredient” and/or the “prophylactic or therapeutic agents”.

In some embodiments, the method comprises local or systemic delivery of β-caryophyllene and one or more statins as prophylactic or therapeutic agents with or without excipients for improved bone healing and regeneration.

In some embodiments, the pharmaceutical composition of the invention further comprises at least one additional active ingredient with or without excipients.

In some embodiments, the pharmaceutical composition further comprises at least one ingredient comprising or characterized by having angiogenic properties. In some embodiments, the at least one additional angiogenic ingredient comprises a nitric oxide donor. In some embodiments, a nitric oxide donor comprises arginine. In some embodiments, the at least one additional angiogenic ingredient improves solubility of a statin, as disclosed herein.

In some embodiments, β-caryophyllene has a complementary synergetic effect with the statin. In some embodiments, β-caryophyllene and the at least one additional active ingredient have a complementary synergetic effect.

In some embodiments, the method comprises administering to the subject a synergistically effective amount of a pharmaceutical composition comprising β-caryophyllene and a statin.

In some embodiments, there is provided a method for treating a bone-related disease or a disorder associated therewith, comprising administering to a subject in need thereof, a therapeutically effective amount of β-caryophyllene and a therapeutically effective amount of a statin. In some embodiments, β-caryophyllene is formulated in a first pharmaceutical composition, and statin is formulated in a second pharmaceutical composition. In some embodiments, the first pharmaceutical composition and the second pharmaceutical composition are administered simultaneously or sequentially.

In some embodiments, there is provided a combination of β-caryophyllene and a statin, for use in the treatment of a bone-related disease or a disorder associated therewith.

In some embodiments, β-caryophyllene is formulated within a first pharmaceutical composition and the statin is formulated within a second pharmaceutical composition.

In some embodiments, there is provided a method for increasing the bioavailability of β-caryophyllene and/or the statin, in a subject in need thereof.

In some embodiments, there is provided a method for increasing the therapeutic activity of β-caryophyllene, in a subject in need thereof, comprising administering to the subject a therapeutically and/or synergistically effective amount of a statin.

In some embodiments, there is provided a method for increasing the therapeutic activity of a statin, in a subject in need thereof, comprising administering to the subject a therapeutically and/or synergistically effective amount of a β-caryophyllene.

Further embodiments of the invention are directed to methods of treating or preventing bone and joint diseases, conditions, or symptoms, and in particular bone healing and regeneration following bone fracture or dental sinus lift and/or dental grafts.

In some embodiments, the herein provided synergistic combination of β-caryophyllene and a statin effectively reduces the therapeutically effective amount of any one of β-caryophyllene and a statin when provided individually.

In some embodiments, any one of the herein disclosed pharmaceutical composition(s) and/or formulation(s) is co-administered with one or more additional active ingredient(s), to the subject. Co-administered comprises simultaneous administration (e.g., provided individually at the same time) or sequential administration (e.g., provided at different time points).

The compositions, or each ingredient, may be administered to a subject, human or animal, by any method known to a person skilled in the art, such as topically, parenterally, paracancerally, transmucosally, transdermally, intramuscularly, intravenously, intradermally, subcutaneously, intraperitonealy, intraventricularly, intracranially, intravaginally or intratumorally.

The compositions, or each ingredient, may be administered to a subject, human or animal, during surgery separately or as a part of any implantable device or system by any method known to a person skilled in the art.

In some embodiments the composition, or each ingredient, is administered directly into the bone fracture or to the dental graft.

In some embodiments, the compositions, or each ingredient, may be added to food. In some embodiments the food is pet food. In some embodiments the food is edibles such as gummies.

The composition, or each ingredient, may be packed in liposomes or emulsions, nanoparticles or micelle.

The absorption of the ingredients may be increased by combining the use of hostile biophysical environments with the use of penetrating agents.

The compositions of the present invention may include additional adjuvants, which are ingredients that are not physiologically active but serve to enhance the properties of the final composition. For example, the compositions of the present invention may include excipients. The compositions of the present invention may include lubricating agents, wetting agents, emulsifying, and suspending agents or preserving agents.

The compositions of the present invention may be formulated in any pharmaceutically acceptable vehicle that does not interact adversely with the active ingredients. Compositions of the present invention may be formulated in water- or oil-based vehicles.

The compositions of the present invention may have a pH of between about 3 and about 8.

According to some embodiments, the composition is administered orally, wherein a unit dosage form used may comprise tablets, capsules, lozenges, chewable tablets, suspensions, emulsions, and the like. Such unit dosage forms comprise a safe and effective amount of the desired compound, or compounds. The acceptable carrier suitable for the preparation of unit dosage forms for peroral administration are well-known in the art.

The administered dosage form may include predefined release profile. In one embodiment, the administered dosage form of the present invention is an extended release formulation. The administered dosage form of the present invention may comprise slow-release means. The administered dosage form of the present invention may comprise immediate release means. The administered dosage form may be formulated according to the desired release profile of the active ingredients, as known to one skilled in the art.

The compositions may comprise liquid solutions, emulsions, suspensions, and the like. The pharmaceutically acceptable carriers suitable for preparation of such compositions are well known in the art.

Compositions for use in the methods of this invention may comprise solutions or emulsions, which, in some embodiments, are aqueous solutions or emulsions comprising a safe and effective amount of β-caryophyllene and a statin, and optionally, other compounds.

### General

As used herein the term “about” refers to ±10%.

The terms “comprises”, “comprising”, “includes”, “including”, “having” and their conjugates mean “including but not limited to”.

The term “consisting of means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments unless the embodiment is inoperative without those elements.

The descriptions of the various embodiments of the present invention have been presented for purposes of illustration but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

Various embodiments and aspects of the present invention as delineated hereinabove and as claimed in the claims section below find experimental support in the following examples.

As used herein, the terms “synergy” or “synergistic” or “synergetic effect” interchangeably refer to the combined effects of two active agents that are greater than their additive effects. Synergy can also be achieved by producing an efficacious effect with combined inefficacious doses of two active agents.

Other terms as used herein are meant to be defined by their well-known meanings in the art.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments unless the embodiment is inoperative without those elements.

### EXAMPLES

Generally, the nomenclature used herein, and the laboratory procedures utilized in the present invention include molecular, biochemical, microbiological, and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds.) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, CA (1990); Marshak et al., “Strategies for Protein Purification and Characterization-A Laboratory Course Manual” CSHL Press (1996); all of which are incorporated by reference. Other general references are provided throughout this document.

### Example 1

### The Formulae

Fourteen different daily formulae would be prepared, as detailed in Table 1.

TABLE 1
The formulae

| | Component | | | | |
|---|---|---|---|---|---|
| | CBD | β-caryophyllene | Lovastatin | Simvastatin | Arginine |
| Composition | (10 mg/kg) | (10 mg/kg) | (1 mg/kg) | (1 mg/kg) | (200 mg/kg) |
| 1. Control | − | − | − | − | − |
| 2. Control | + | − | − | − | − |
| 3. Control | − | + | − | − | − |
| 4. Control | − | − | + | − | − |
| 5. Control | − | − | − | + | − |
| 6. Control | − | − | − | − | + |
| 7. G7 | + | + | − | − | − |
| 8. G8 | + | − | + | − | − |
| 9. G9 | + | − | − | + | − |
| 10. G10 | + | − | − | − | + |
| 11. G11 | − | + | + | − | − |
| 12. G12 | − | + | − | + | − |
| 13. G13 | − | + | − | − | + |
| 14. G14 | − | − | + | + | − |
| 15. G15 | − | − | + | − | + |
| 16. G16 | + | + | + | − | − |
| 17. G17 | + | − | + | + | − |
| 18. G18 | − | + | + | + | − |
| 19. G19 | + | − | − | + | + |
| 20. G20 | − | + | + | − | + |
| 21. G21 | − | − | + | + | + |
| 22. G22 | + | + | + | + | − |
| 23. G23 | + | + | + | − | + |
| 24. G24 | + | + | − | + | + |
| 25. G25 | + | − | + | + | + |
| 26. G26 | − | + | + | + | + |
| 27. G27 | + | + | + | + | + |

The formulae will also compose inert ingredients for improved bioavailability and to control the release time of the ingredients.

In formulae containing CBD and β-caryophyllene, half doses will be tested as well. In formulae containing lovastatin and simvastatin, half doses will be tested as well.

### Example 2

### Repeated Intraperitoneal Administration

The toxicity of a composition comprising of 18 mg/kg/day of β-caryophyllene or CBD as an activator of the endocannabinoid system, and 4.5 mg/kg/day of one statin, either Lovastatin or Simvastatin, was determined along 14 days. No adverse events were observed (data not shown).

### Example 3

### Optimization of Ingredients Ratios

The trial includes approximately six animal groups for six different ratios of the active pharmaceutical ingredients; i.e., β-caryophyllene, Lovastatin, Simvastatin, CBD, and their combinations.

The trial is performed as described in Example 2 (acute oral administration).

The trial is performed on Sprague Dawley male rat (n=3/group).

A single dose is provided per os (PO), intravenously (IV) or to the bone periosteum at t=0.

The active ingredients' pharmacokinetics (Pk) is examined: 1, 4, 8 and, 12 hours following a single administration.

The target is to optimize the ratio between the various active pharmaceutical ingredients, and to assess potential toxicity or intolerability.

### Example 4

### Oral Chronic Administration

The trial is performed on Sprague Dawley male rat (n=12/group, either sex; >12 weeks old; >250 grams) using a Tibial Tuberosity Advancement (TTA) model, an orthopedic procedure to repair deficient cranial cruciate ligaments. The cranial cruciate ligament (CrCL) stabilizes the knee joint (the stifle joint) and limits the tibia from sliding forward.

A single dose of each formula mentioned in Table 1 is provided PO, intravenously (IV) or to the bone periosteum at t=0, and every 24 h for 30 days. The trial will last 56 days.

The objective of the trial is to determine the effect on bone healing of a candidate test article when administered orally.

A physical examination comprising recordation of the general condition of the animals is performed before entry into the study. All animals are in apparent general good health as determined by the physical examination prior to being cleared for study participation. Prior to necropsy, a similar physical examination is repeated on all animals completing the study.

Under general inhalational anesthesia and utilizing strict aseptic technique, an incision is made on the lateral aspect of the femur, extending along its entire length. Surrounding tissues are carefully dissected to completely expose it. A polyacetal neutralization plate is fitted to the bone and used as a template to locate (and mark) the sites for the screw holes. Bi-cortical pilot holes, 1.1-mm in diameter, are created for each screw. The plate is repositioned on the bone, and 1.5-mm diameter by 10 mm long, self-tapping, 316L stainless steel, cortical screws are used to securely fix the plate to the femur. After the plate is secured, an osteotomy is created in the mid-span of the plate using a 1.1-mm cutting burr. Constant irrigation with sterile irrigation solution is used during creation of the ostectomy to avoid heat damage to the tissues. Debris are cleared from the osteotomy site with an irrigation solution wash and suction. The plate is then removed, the TA applied to encircle the osteotomy, and the plate then re-applied. The wound is sealed or closed in layers using appropriately sized suture material in a simple continuous pattern. The skin is closed with appropriately sized suture material, placed in a subcuticular pattern. An identical procedure is then performed on the opposite femur.

Radiography of the femurs is obtained as soon as post-surgery permits (e.g., practical after surgery). Clinical Observations are performed daily for the first 14 days, and then weekly.

Radiographic images of the operated and unoperated femurs are obtained for each animal within 3 days after surgery and on days 14, 28, 42 and 56±3. The rats are examined in ventro-dorsal [VD a/k/a anterior-posterior (AP)] and lateral projections. Each projection contains an embedded step wedge densitometry scale to provide a density calibration for radiographic assessment of healing. The scoring system presented below is used to qualitatively assess the healing of the defect.

The Radiographic Scoring System is detailed in Table 2.

TABLE 2
Radiographic Scoring System.

| Score | Description |
|---|---|
| 0 | No or minimal callus; trace radio dense material in defect |
| 1 | Mineralized callus spanning the defect, yet not bridged. |
| | Flocculent radio density and incomplete bridging of defect |
| 2 | Mineralized callus spanning the defect, |
| | bridging of the defect at 1+ location |
| 3 | Bridging callus remodeling at cis and trans |
| | cortices towards cortical bone; parent cortex visible |
| 4 | Cortical bridging across the defect regenerating marrow space; |
| | one cortex obscured by new bone |
| 5 | Bridging of the defect by uniform new bone, |
| | cut ends of cortex not seen |

All animals are euthanized, and the implanted femurs collected for histological evaluation on Day 56 after surgery. After euthanasia, the operated limb is carefully disarticulated at the hip and knee. Muscle tissues are carefully dissected, avoiding disruption of the callus and defect site. The femurs are placed in 10% neutral buffered formalin for a period of at least four days before being processed for examination.

The plasma concentrations of β-caryophyllene, Lovastatin and Simvastatin, and CBD are examined 8 and 12 hours following the first administration, and every 4 days thereafter until the end of the trial.

The trial is repeated with various concentrations and ratios of the active pharmaceutical ingredients; i.e., β-caryophyllene, Lovastatin, Simvastatin, and CBD.

### Example 5

### Local Administration

The purpose of the study was to determine the efficacy of 4 tested groups on healing of surgically induced pseudo-fracture. Pseudo-fractures were created by making a trough on one surface of the femoral shaft through one side of the cortex. The compositions were administered locally by daily injection. Animals: Naive male Sprague Dawley rats (SD rats), 12 weeks old, around 350 g. Right leg was treated while the left leg serves as a control (vehicle). Groups included: β-caryophyllene: Lovastatin (G1−B+L); β-caryophyllene: Simvastatin (G2−B+S), CBD: Lovastatin (G3-C+L) and CBD: Simvastatin (G4−C+S). All combinations included β-caryophyllene or CBD to Lovastatin or Simvastatin weight per weight ratios of 10:1.

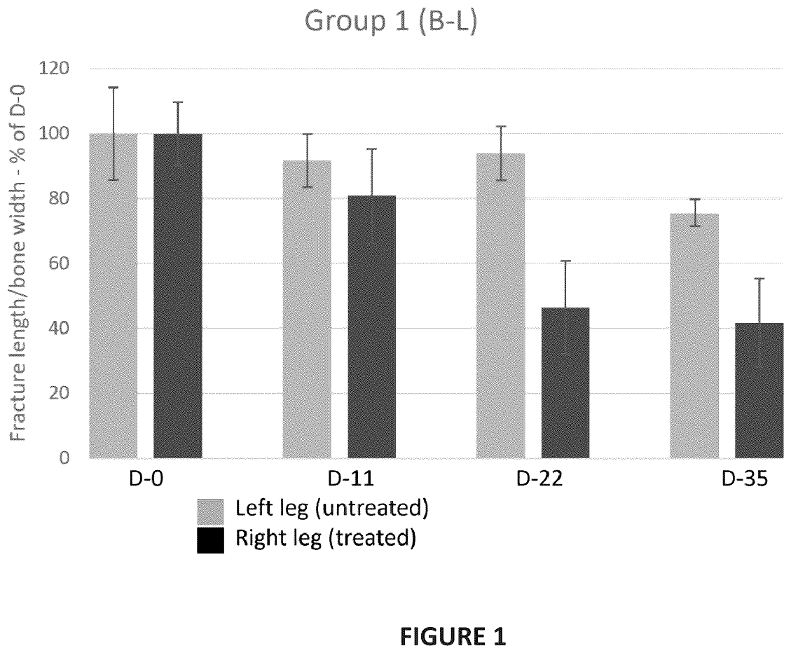

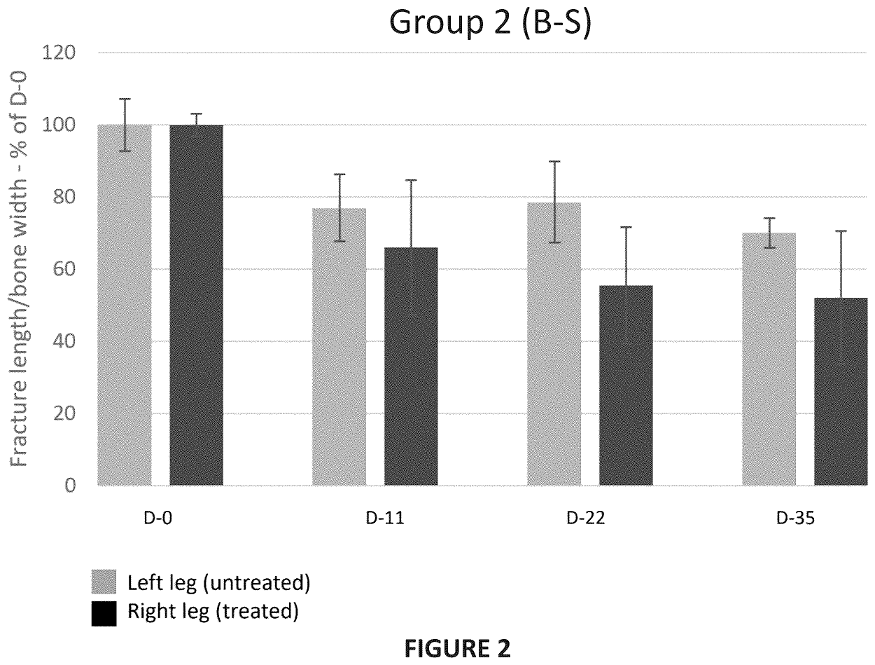

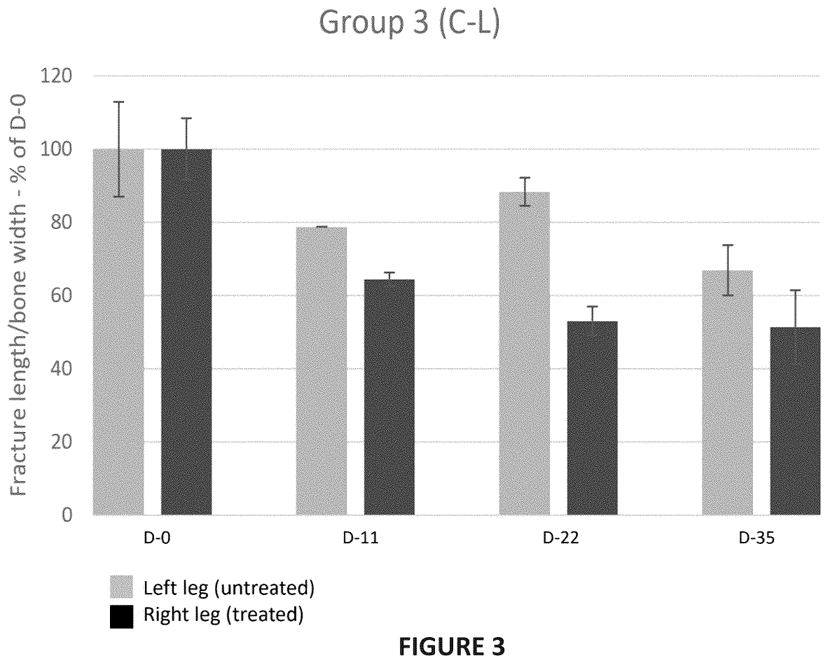

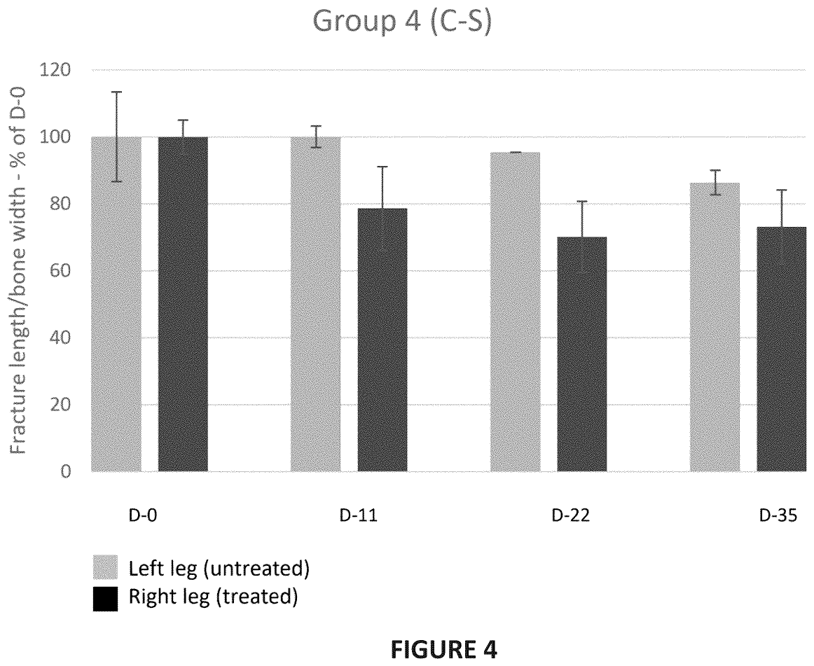

Animals' body weight did not change significantly along the trial. In all groups, improved healing was observed comparing to vehicle alone. The greatest improvements were observed for fractures treated with β-caryophyllene and Lovastatin; by ˜12% at Day 11, by ˜51% at Day 22, and by ˜45% at Day 35 (FIG. 1). Significant improvement was observed for fractures treated with β-caryophyllene and Simvastatin; by ˜14% at Day 11, by ˜29% at Day 22, and by ˜26% at Day 35 (FIG. 2). Significantly less improvement was observed for compositions containing CBD. Improved healing by ˜18% at Day 11, by ˜40% at Day 22, and by ˜23% at Day 35 for CBD with Lovastatin and improved healing by ˜21% at Day 11, by ˜27% at Day 22, and by ˜15% at Day 35 for CBD and Simvastatin (FIGS. 3-4, respectively).

### Example 6

### Doses for Local Administration

A clinical trial is conducted to determine the dose ranges for the combination of the active pharmaceutical ingredients; i.e., CBD, β-caryophyllene, Lovastatin and Simvastatin.

Fifty (50) human patients suffering from bone fracture are treated with local different dose ranges according to the results of Example 3 (ratios) and Example 5 (local administration). The doses are administered in surgery.

Post-surgery examination is performed as detailed in Example 4.

### Example 7

### Maximum load (N) Following 35 Treatment Days of Femoral Bone Fracture

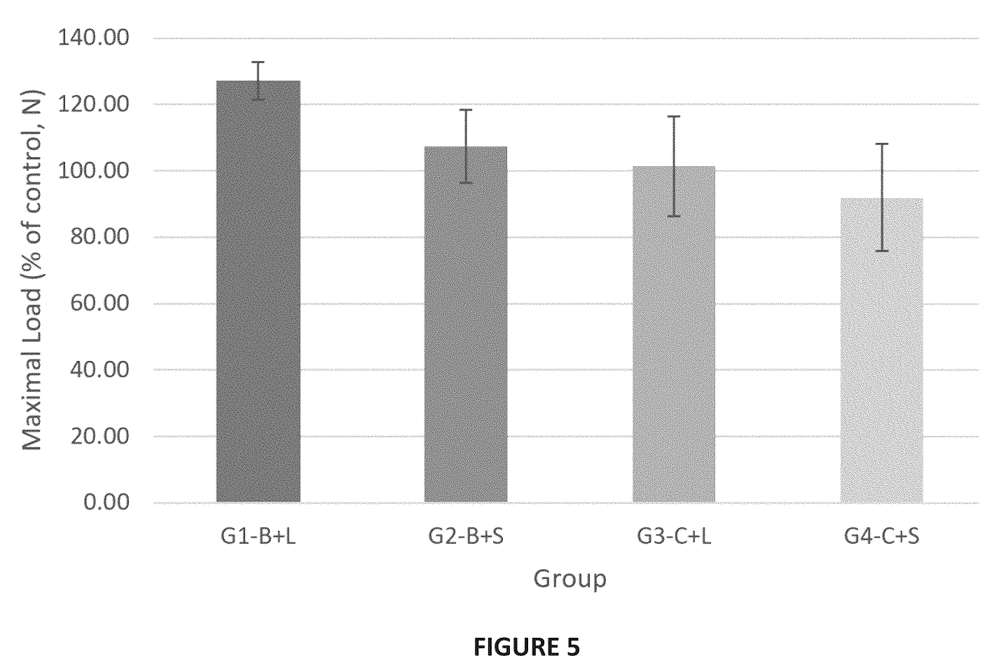

Bone maximum load (N) following 35 treatment days of femoral bone fracture was measured in 4 groups: β-caryophyllene and Lovastatin (G1−B+L), β-caryophyllene and Simvastatin (G2−B+S), CBD and Lovastatin (G3−C+L), and CBD and Simvastatin (G4−C+S). An improvement of ˜27% was observed for the G1−B+L treatment group compared to vehicle-treated (FIG. 5).

### Example 8

### Stiffness (N/mm) Following 35 Treatment Days of Femoral Bone Fracture

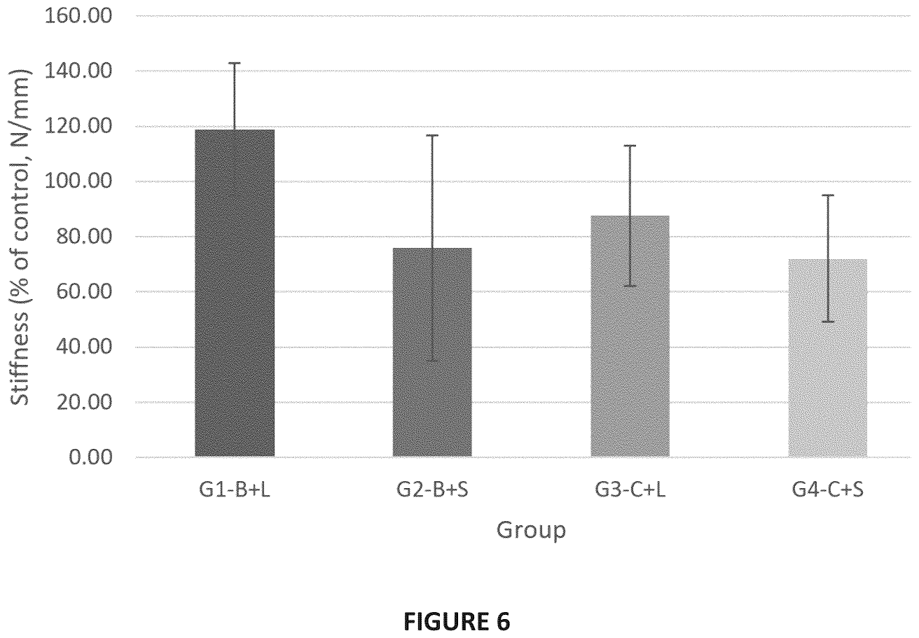

Bone stiffness (N/mm) following 35 treatment days of femoral bone fracture was measured in 4 groups: β-caryophyllene and Lovastatin (G1−B+L), β-caryophyllene and Simvastatin (G2−B+S), CBD and Lovastatin (G3−C+L), and CBD and Simvastatin (G4−C+S). An improvement of ˜19% was observed for the B+L treatment group compared to vehicle-treated (FIG. 6).

### Example 9

### Energy to Maximal Load (mj) Following 35 Treatment Days of Femoral Bone Fracture

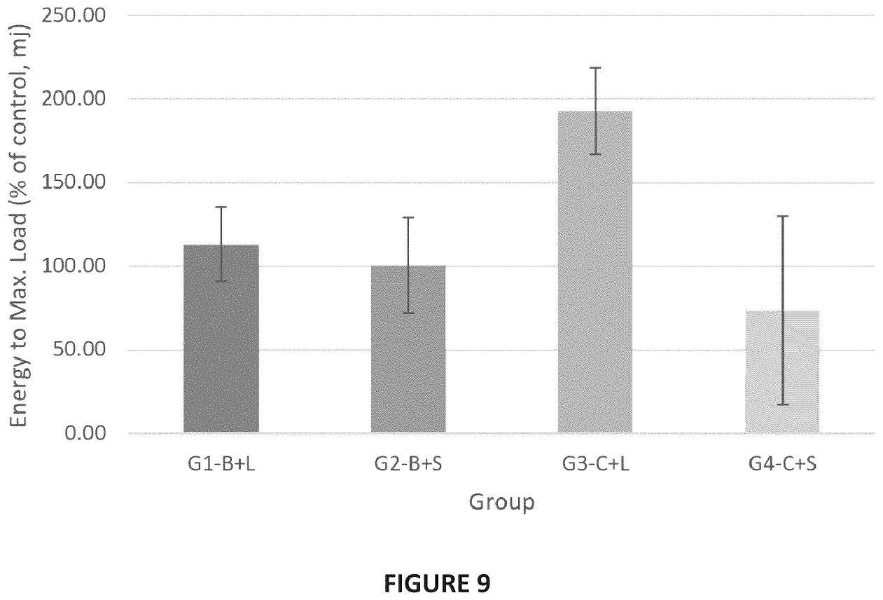

Bone energy to maximal load (mj) following 35 treatment days of femoral bone fracture was measured in 4 groups: β-caryophyllene and Lovastatin (G1−B+L), β-caryophyllene and Simvastatin (G2−B+S), CBD and Lovastatin (G3−C+L) and CBD and Simvastatin (G4−C+S). An improvement of ˜14% for the B+L treatment group, was observed, compared to vehicle-treated (FIG. 9). C+L treatment also provided a substantial improvement compared to the vehicle-treated group.

### Example 10

### Bone Quality

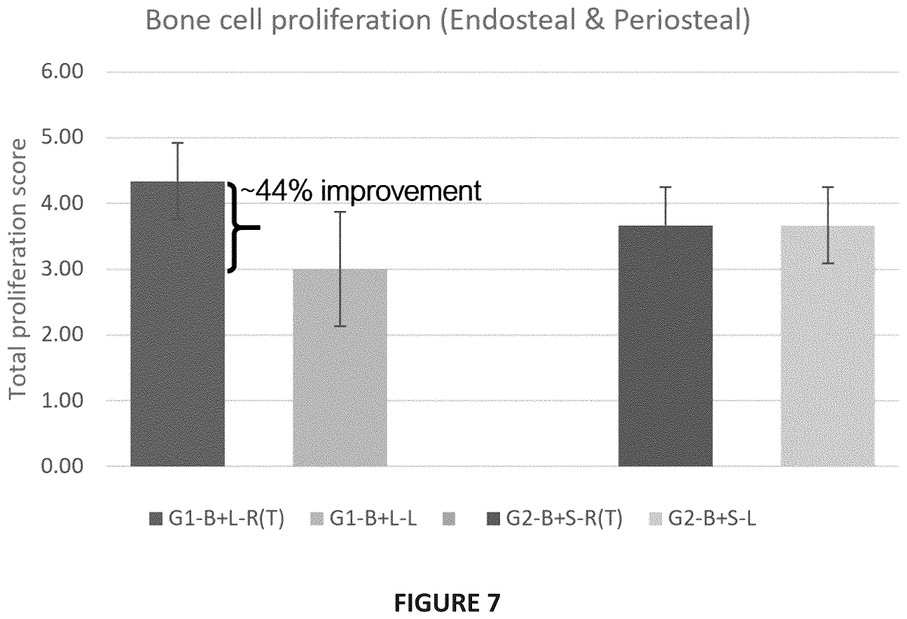

Bone quality was examined after 35 treatment days. For the G1−B+L group, cell proliferation was improved by >44% compared to untreated bone. No change in cell proliferation was observed for the G2−B+S group (FIG. 7), following decalcification.

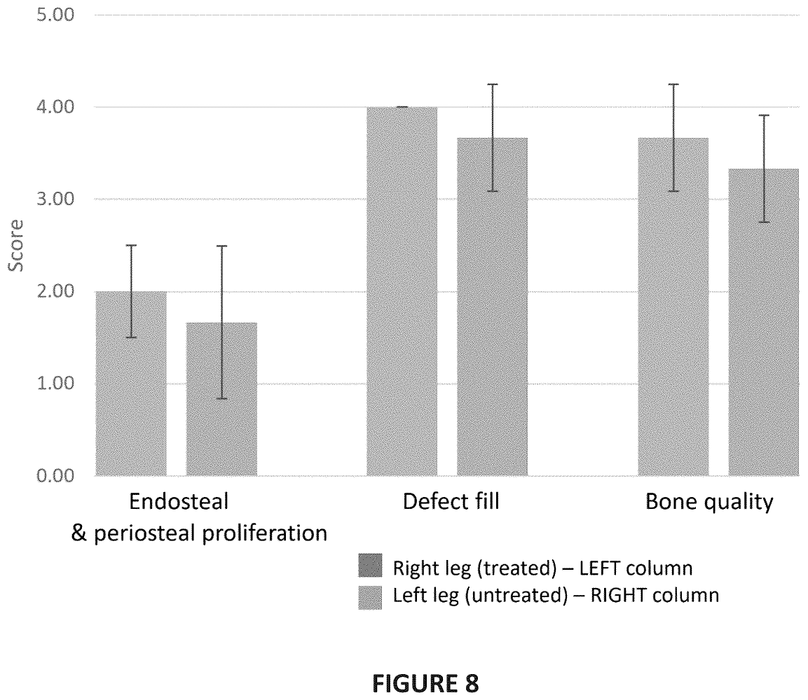

Bone endosteal & periosteal proliferation, defect fill and bone quality were evaluated following 35 treatment days of femoral bone fracture with β-caryophyllene (none-decalcification). Significant improvement was observed in: endosteal and periosteal proliferation, defect fill, and bone quality (FIG. 8).

### Example 11

### In Vitro Cytotoxicity

In vitro cytotoxicity & proliferation assay (basal) are being performed on human osteoblasts and stem cells monolayers.

Human primary osteoblast cells are maintained in a 37° C. humidified incubator with 5% CO₂ atmosphere. Cells are cultured in Primary Osteoblast Media containing Primary Osteoblast Culture Supplement and 1% of the penicillin/streptomycin stock solution.

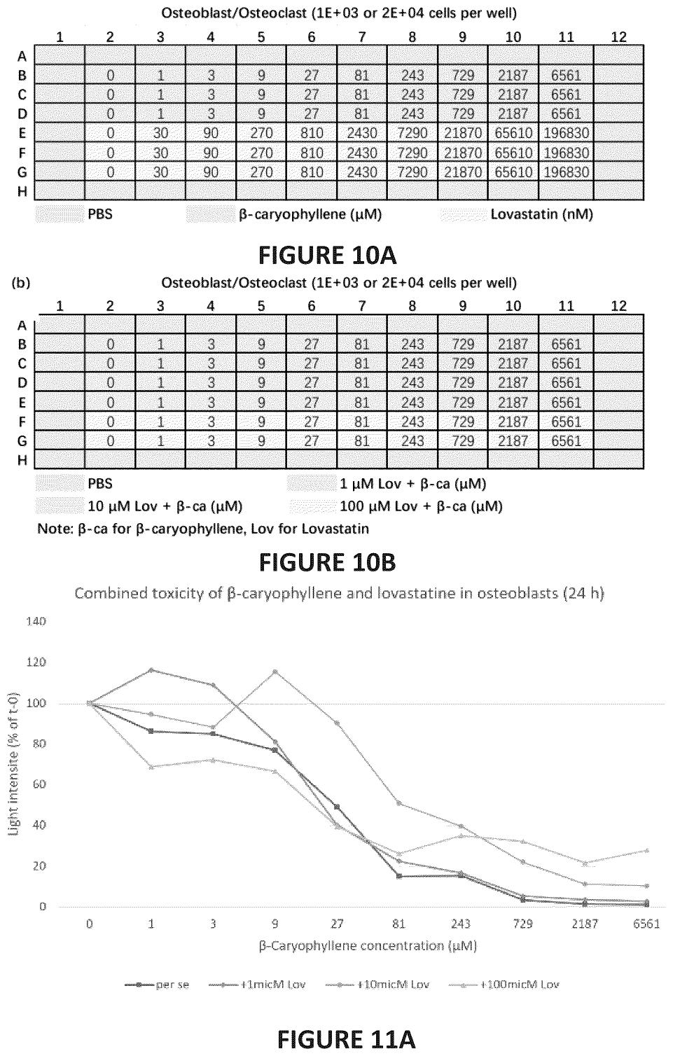

As a human primary osteoclast cell is unable to proliferate, MUTZ-3, a human cell line model for osteoclast differentiation is also being used. MUTZ-3 cells are maintained in a 37° C. humidified incubator with 5% CO₂ atmosphere. Cells are cultured in Dulbecco's modified Eagle's Medium (DMEM) supplemented with 10% foetal calf serum and 1% of the penicillin streptomycin stock solution. Plate layout is shown in FIG. 10.

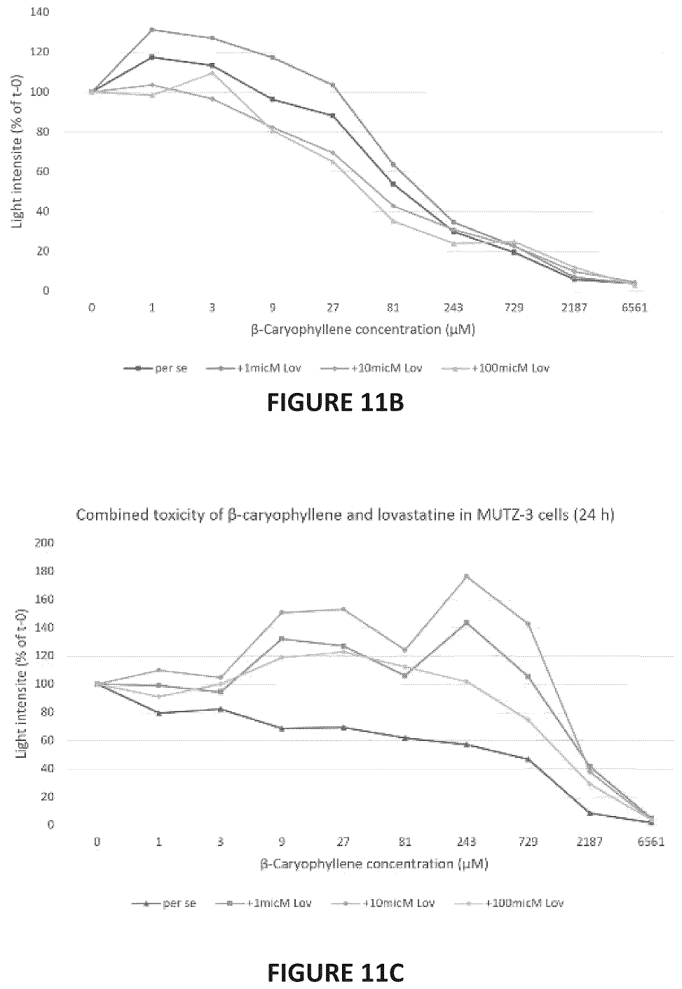

Light intensity (fluorescence intensity at 485-500 nm_{Ex}/520-530 nm_{Em}) was measured on osteoblasts culture with β-caryophyllene concentration from 0 to 6,561 μM with lovastatin at concentrations of 0, 1, 10 and 100 μM after 24 hours (FIG. 11A) and 96 hours (FIG. 11B). Addition of lovastatin, at all concentrations, did not affect β-caryophyllene's cytotoxicity. β-caryophyllene does not present a significant cytotoxic effect for concentration up to about 27 μM.

Light intensity (fluorescence intensity at 485-500 nm_{Ex}/520-530 nm_{Em}) was measured on MUTZ-3 culture with β-caryophyllene concentration from 0 to 6,561 μM with lovastatin at concentrations of 0, 1, 10 and 100 μM after 24 hours (FIG. 11C) and 96 hours (FIG. 11D). Addition of lovastatin, at all concentrations, did not affect β-caryophyllene's cytotoxicity. β-caryophyllene does not present significant cytotoxic effect for concentration up to about 27 μM.

### Example 12

### Differentiation of hMSC (Human Mesenchymal Stem Cells) into Osteoblasts

Human mesenchymal stem cells (hMSC) are maintained in a 37° C. humidified incubator with 5% CO₂ atmosphere. Cells are cultured in Primary Mesenchymal Stem Cells Serum-Free Media and Primary Osteoblast Media containing Primary Mesenchymal Stem Cell Culture Supplement, and 1% of the penicillin streptomycin stock solution (growth medium). hMSC differentiation into osteoblast was induced by OriCell® Human Related Stem Cells Osteogenic Differentiation Kit (Cyagen, China; osteogenic medium).

Five thousand (5,000) hMSC/well of are seeded in 24-well cell culture plate with growth medium to expand to suitable density. The cells are washed with PBS (Phosphate Buffered Saline), which is subsequently changed to osteogenic medium containing specific inflammatory cytokines. The medium is changed twice a week for 21 days. The candidate cytokines include: interleukin-1beta (IL-1β), tumor necrosis factor alpha (TNF-α), interferon gamma (IFN-γ), IL-6, IL-17A, IL-10, IL-8, transforming growth factor beta 1 (TGF-β1) and vascular endothelial growth factor A (VEGF-A).

The differentiation effect is evaluated by the matrix mineralization, and measured and quantified by Alizarin Red staining. The best inflammatory cocktail is defined as the single inflammatory cytokine or their combinations that induce the most matrix mineralization.

Under the best inflammatory cocktail, expression level of osteoblastic marker genes are evaluated, including Cbfa-1, collagen I, OCN, OPN, osteonectin, Runx2, ALP, and BMP-4. The expression level is measured by quantitative PCR (qPCR). At the same time, secretion profiles of pro/anti-inflammatory proteins and growth factors expression are measured by enzyme linked immunosorbent assay (ELISA), including IFN-γ, IL-8, Prostaglandin E2 (PGE2), IL-10, TGF-β (transforming growth factor), IL-1RA, KGF (Keratinocyte growth factor)/FGF-7 (fibroblast growth factor), ANG-1 and TSG-6 (tumor necrosis factor-inducible gene 6 protein).

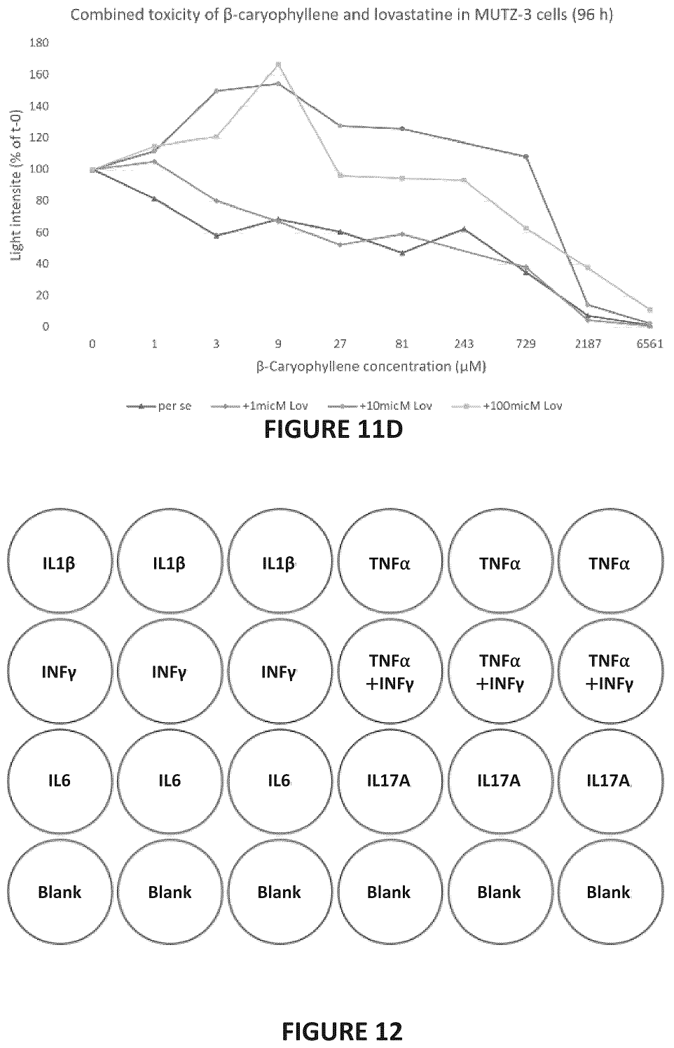

Plate layout (FIG. 12) represent the study course. First round hMSC differentiation to test inflammatory cytokines in 24-well plate to define best cytokine cocktail. Second round after best cytokine cocktail stimulation in 6-well plate, harvest cell pellet to conduct qPCR and supernatant to ELISA. The composition of β-caryophyllene and lovastatin indicates modulation of the cytokines and their expression.

### Example 13

### Ex-Vivo Observations: Calcification of Bone Slices

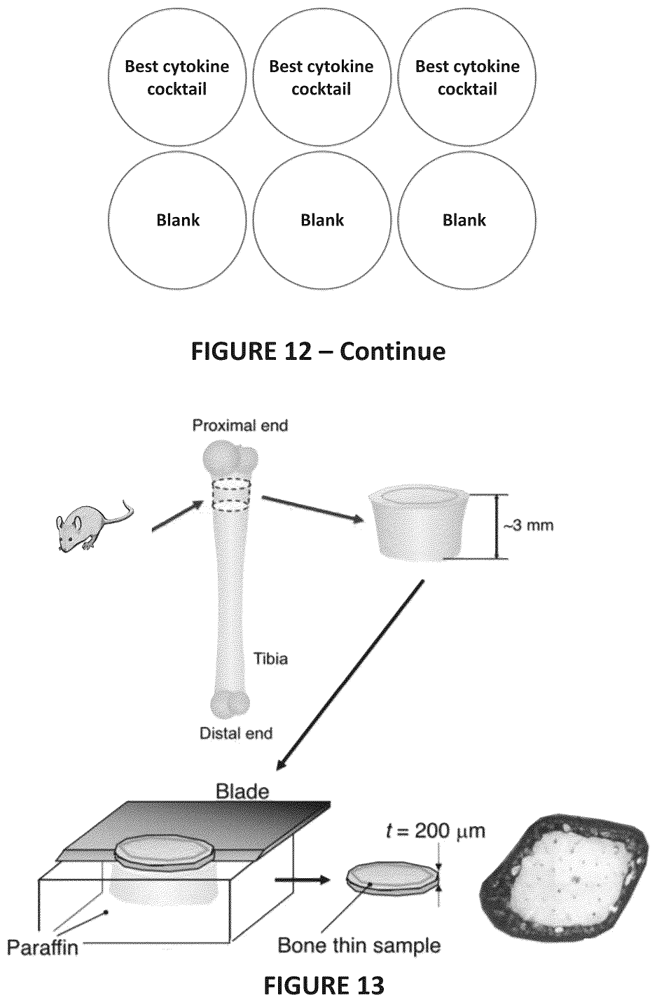

Bone explants are obtained from the mice tibia (FIG. 13, following Maeda et al., 2018; Journal of Biomechanics). Animals are sacrificed using CO₂ gas. Both left and right legs are sterilized with 70% ethanol, and the tibia is excised within a clean bench. A bone slice, with a thickness of about 3 mm, is cut out from a plane perpendicular to the tibial axis, at about 3-7 mm distance from the proximal end of the tibia. The slice is immediately embedded in paraffin, and placed in a holder of a micro-slicer filled with phosphate buffered saline (PBS). A thin slice, with a thickness of about 200-2,000 μm, is cut out from the middle part of the explant. The thin slice is washed for 5 seconds in PBS supplemented with 10% penicillin-streptomycin and kept until subsequent culture experiment in a culture medium consisting BGJb supplemented with 10% fetal bovine serum, 10% penicillin-streptomycin, 75 μg/ml ascorbic acid, 5 mM β-glycerophosphate and 10⁻⁷ M dexamethasone.

At up to 35 days old, there is a thin layer of calcified tissue already present in the outer edge of the tibia, which accounts for approximately 10-40% of the cross-sectional area of the tibia on average.

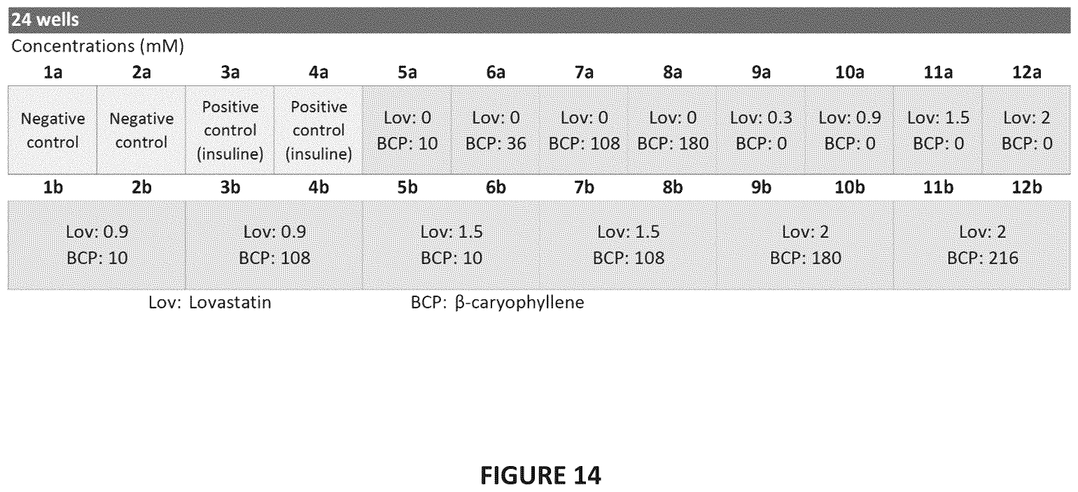

To evaluate the effects of β-caryophyllene and lovastatin on the calcification of the bone specimens during the culture, bone specimens are placed in wells and incubated with β-caryophyllene (0-216 mM) and lovastatin (0-2 mM) (FIG. 14). Fifty (50) μg/mL of insulin, that is known to stimulate new bone formation through activation of the insulin-like growth factor 1 (IGF-1) receptor, served as a control. Bone specimens are analyzed using Alzarin Red S staining or similar.

The calcified area obtained is evaluated every 24 h, up to about 30 days. The calcified area in bone specimens incubated with low β-caryophyllene and lovastatin concentrations remained at the levels of unstretched control samples whereas the area in medium β-caryophyllene and lovastatin concentrations was markedly higher than those of unstretched samples, but lower in high concentrations. The area is growing along time, where the faster calcification rate is observed up to 10 days.

### Example 14

### Ex-Vivo Observations: Quantitative Assessment of Viability

To monitor the viability of the bone specimens over the tested time frame the concentration of lactate dehydrogenase (LDH), is analyzed in the culture supernatant. LDH is released in response to cell membrane damage and demonstrated a significant increase of optical density values. In order to quantitatively assess the degree of cell damage and cell survival of cells in the bone tissue, LDH is measured on day 0 one hour after incubation and every third day. At the end of the trial, bone specimens are treated with 1% (v/v) Triton X-100 in order to force lysis of residual cells and measure their LDH content. LDH assay is performed according to manufacturer's instructions. Samples are transferred to a 96-well plate with reaction mixture and the optical density at 490 nm wavelength is measured. An initial peak of LDH is observed probably due to the ex-plantation process, as well as sawing and drilling process of the bone tissue. In the later stages LDH concentrations reached a steady state at a low level in the tested time frame.

Alkaline phosphatase activity (ALP) as an indicator of osteoblast activity is determined in the supernatant of the bone specimens over time. ALP assay ss performed to assess the osteogenic activity of the bone tissue throughout the trial. ALP activity is measured using an ALP assay kit. Measurements are performed according to manufacturer's instruction and the optical density is measured, using Tecan Infinite® 200 PRO. Measurements are performed in technical duplicates.

Similar to the observations for LDH, the ALP activity is increased during the first week significantly followed by a decrease and finally stayed stable until the end of the trial. The increased levels of ALP at the initial time points seem to be associated with the bone tissue processing methods used to set up the model.

### Example 15

### Ex-Vivo Observations: DNA Quantification

To quantify the number of cells in the bone specimens the amount of DNA on day 7 and day 28 after culturing was analyzed. Bone cylinders are drilled out of bone slices after 7 and 28 days of culturing. After placing each cylinder in a 2 mL Eppendorf tube, 1 mL of nuclease free water is added and the cylinder undergoes 3 freeze and thaw cycles at −80° C. and room temperature. Subsequently the tubes are sonicated twice for 15 seconds at 20 kHz with an amplitude of 12 microns. After sonication samples are stored in −20° C. in an Eppendorf tube until use. Samples are quantified using Quant-iT PicoGreen dsDNA assay kit according to manufacturer's instruction. Measurements were performed using Tecan Infinite® 200 PRO. DNA quantification is performed following incubation with β-caryophyllene and lovastatin, as detailed in previous examples.

DNA amount is increased significantly from day 7 to day 28 with the increase in cell numbers and regeneration process of early state within the bone sections throughout this time frame.

### Example 16

### Ex-Vivo Observations: Cellular Morphology

Confocal laser scanning microscopy (CLSM) images of Calcein-AM stained bone specimens are taken on day 7 and on day 28 to gain a deeper insight into the cellular morphology in the 3-dimenisonal bone sections. CLSM images document a higher density of vital cells on day 28 compared to day 7.

### Example 17

### Ex-Vivo Observations: Cellular Morphology

Bone specimens are stained with Hoechst nuclear stain to depict all cells in the bone section. Overlay images show a high number of viable cells on day 7 and an increased number of cells on day 28.

While the present invention has been particularly described, persons skilled in the art will appreciate that many variations and modifications can be made. Therefore, the invention is not to be construed as restricted to the particularly described embodiments, and the scope and concept of the invention will be more readily understood by reference to the claims, which follow.

1.-10. (canceled)

11. A method of treating a subject afflicted with a bone-related disease or a disorder associated therewith, comprising administering to said subject a therapeutically effective amount of a pharmaceutical composition comprising β-caryophyllene and a statin.

12. The method of claim 11, wherein said bone-related disease or a disorder associated therewith comprises a bone fracture.

13. The method of claim 11, wherein said treating comprises: reducing fracture length per bone width, increasing maximal load, increasing stiffness, or any combination thereof, of a bone of said subject.

14. The method of claim 11, wherein said treating comprises increasing number of bone proliferating cells, rate of bone cell proliferation, or both, in said subject.

15. The method of claim 11, wherein said treating comprises increasing: endosteal and periosteal proliferation, defect fill, bone quality, or any combination thereof, in a bone of said subject.

16. The method of claim 11, wherein said administering comprises locally administering.

17. The method of claim 11, wherein said administering comprises intraoperative administration.

18. The method of claim 11, wherein said pharmaceutical composition comprises said β-caryophyllene and said statin in a w/w ratio ranging from 10:1 (w/w) to 1:10 (w/w).

19. The method of claim 11, wherein said statin is a hydrophilic statin or a lipophilic statin.

20. The method of claim 11, wherein said statin is selected from the group consisting of: lovastatin, simvastatin, fluvastatin, rosuvastatin, pravastatin, pitavastatin, and any combination thereof.

21. The method of claim 11, wherein said statin is lovastatin or simvastatin.
